(19) United States
(12) Patent Application Publication
(10) Pub. No.: US 2012/0232528 A1
(43) Pub. Date: Sep. 13, 2012

(54) DELIVERY SYSTEM WITH INCREMENTAL MARKERS

(75) Inventors: Erik D. Eli, Redwood City, CA (US); Laura M. Kalvass, Mountain View, CA (US)

(73) Assignee: ABBOTT CARDIOVASCULAR SYSTEMS INC., Santa Clara, CA (US)

(21) Appl. No.: 13/042,033

(22) Filed: Mar. 7, 2011

(51) Int. Cl. A61M 25/098

(52) U.S. Cl. 604/529

(57) ABSTRACT

The present invention is a catheter or catheter delivery system that incorporates a series of radiopaque markers at discrete, intervals corresponding to units of measurement, such as millimeters, such that structures and blockages within a body lumen can be measured in vitro using the catheter under fluoroscopy. The apparatus and method avoids estimation errors by directly measuring the designated lesion, allowing for improved selection of stents and balloons.

### BACKGROUND

This invention generally relates to intravascular balloon catheters and systems for performing percutaneous transluminal coronary angioplasty (PTCA) and/or stent delivery, and more particularly to a catheter delivery system that can accurately evaluate and measure objects within a body lumen using incremental radiopaque markers as an illuminated ruler.

PTCA is a widely used procedure for the treatment of coronary heart disease. In this procedure, a balloon dilatation catheter is advanced into the patient's coronary artery and the balloon on the catheter is inflated within the stenotic region of the patient's artery to open up the arterial passageway and thereby increase the blood flow there through. To facilitate the advancement of the dilatation catheter into the patient's coronary artery, a guiding catheter having a pre-shaped distal tip is first percutaneously introduced into the cardiovascular system of a patient by the Seldinger technique or other method through the brachial or femoral arteries.

The catheter is advanced until the pre-shaped distal tip of the guiding catheter is disposed within the aorta adjacent the ostium of the desired coronary artery, and the distal tip of the guiding catheter is then maneuvered into the ostium. A balloon dilatation catheter may then be advanced through the guiding catheter into the patient's coronary artery over a guidewire until the balloon on the catheter is disposed within the stenotic region of the patient's artery. The balloon is inflated to open up the arterial passageway and increase the blood flow through the artery. Generally, the inflated diameter of the balloon is approximately the same diameter as the native diameter of the body lumen being dilated so as to complete the dilatation but not over expand the artery wall. After the balloon is finally deflated, blood flow resumes through the dilated artery and the dilatation catheter can be removed.

In a large number of angioplasty procedures, there may be a restenosis, i.e. reformation of the arterial plaque. To reduce the restenosis rate and to strengthen the dilated area, physicians may implant an intravascular prosthesis or “stent” inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is then deflated to remove the catheter and the stent is left in place within the artery at the site of the dilated lesion.

To accurately place the balloon at the desired location, visual markers on the catheter are utilized that are read by machines outside the body. For example, in the case where a balloon catheter is used with an fluoroscope, a radiopaque marker may be observed visually on a screen while the procedure is taking place. In many cases, the markers must be precisely located to ensure accurate placement of the balloon in the affected area.

When performing a procedure such as PTCA, it is crucial that the proper size balloon and stent be utilized. The appropriate sized stent is governed in the radial direction by the diameter of the body lumen at the site of implantation, such that the expanded stent diameter roughly matches the ordinary lumen channel without undue expansion. The proper length of the stent is selected based on the amount and size of the blockage or lesion, which can be a challenge to measure accurately using conventional visual tools. If the stent is too short, it can be ineffective in achieving the desired performance and may require that the patient be subjected to a second procedure to add another stent. If the stent is too long, there is an increased risk of restenosis due to the longer area of injury. Because the accurate determination of the size of the blockage or lesion is critical to the success of the procedure, better methods are needed to determine with high precision the size of the lesion to be addressed by the stent or the balloon.

One of the challenges that is faced when trying to accurately measure a lesion or other arterial blockage is that the standard methods for observing the lesion uses two dimensional technology to measure three dimensional structures. At two dimensional picture cannot show the true length where the vessel is oblique or offset from the normal view of the viewing instrument. Two dimensional imaging lacks the capability to show tortuosity, overlap, suboptimal projections, and individual anatomic variation, leading to poor measurements. In a study by scientists at the University of Colorado Health Sciences Center in Denver, Colo., it was determined that vessel foreshortening in angiographic images using two dimensional technology resulted in an error of up to fifty percent (50%). See Angiographic Views Used For Percutaneous Coronary Interventions, CATHETERIZATION AND CARDIOVASCULAR INTERVENTIONS 64:451-459 (2005).

### SUMMARY OF THE INVENTION

The present invention is a catheter or catheter delivery system that incorporates a series of radiopaque markers at discrete, intervals corresponding to units of measurement, such as millimeters, such that structures and blockages within a body lumen can be measured in vitro using the catheter under fluoroscopy. The apparatus and method avoids estimation errors by directly measuring the designated lesion, allowing for improved selection of stents and balloons.

### BRIEF DESCRIPTION OF THE DRAWINGS

FIG. 1 is an elevated, perspective view of a catheter delivery system of the present invention;

FIG. 2 is an enlarged view of the balloon catheter in a collapsed condition within a body lumen;

FIG. 3 is an enlarged view of a second embodiment of the balloon catheter;

FIG. 4 is an enlarged view of the balloon catheter of FIG. 3 in an expanded condition;

FIG. 5 is an enlarged view of the inner member with a spiral wound radiopaque ribbon wrapped around the inner member having a pitch with a predetermined value; and

FIG. 6 is an enlarged, cross-sectional view of the inner member with a plurality of raised beads spaced along the inner member.

### DETAILED DESCRIPTION OF THE PREFERRED EMBODIMENTS

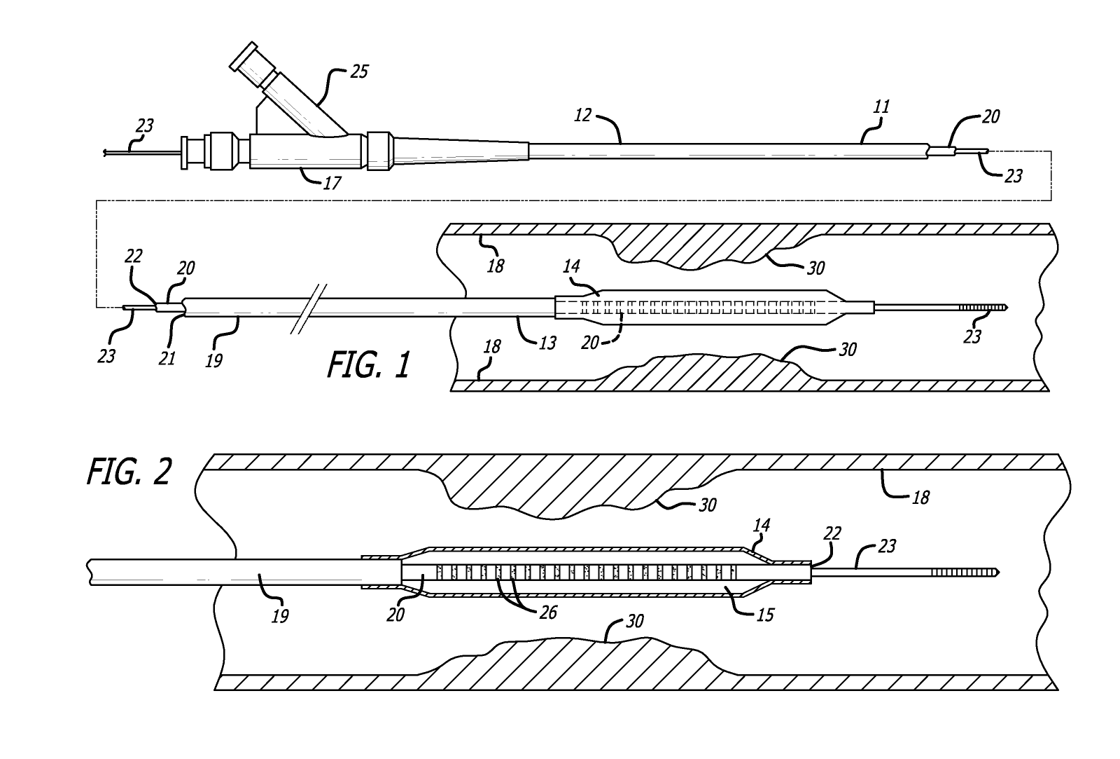

FIG. 1 shows a balloon catheter that can be used to illustrate the features of the invention. The catheter 10 of the invention generally comprises an elongated catheter shaft 11 having a proximal section 12, a distal section 13, an inflatable balloon 14 on the distal section 13 of the catheter shaft 11, and an adapter 17 mounted on the proximal section 12 of shaft 11. In FIG. 1, the catheter 10 is illustrated within a greatly enlarged view of a patient's body lumen 18, prior to expansion of the balloon 14, adjacent the tissue to be acted upon by the balloon.

In the embodiment illustrated in FIG. 1, the catheter shaft 11 has an outer tubular member 19 and an inner tubular member 20 disposed within the outer tubular member and defining, with the outer tubular member, inflation lumen 21. Inflation lumen 21 is in fluid communication with the interior chamber 15 of the inflatable balloon 14. The inner tubular member 20 has an inner lumen 22 extending therein which is configured to slidably receive a guidewire 23 suitable for advancement through a patient's coronary arteries. The distal extremity of the inflatable balloon 14 is sealingly secured to the distal extremity of the inner tubular member 20 and the proximal extremity of the balloon is sealingly secured to the distal extremity of the outer tubular member 19.

The balloon 14 can be inflated by a fluid such as air, saline, or other fluid that is introduced at the port in the side arm 25 into inflation lumen 21 contained in the catheter shaft 11, or by other means, such as from a passageway formed between the outside of the catheter shaft 11 and the member forming the balloon 14, depending on the particular design of the catheter. The details and mechanics of the mode of inflating the balloon vary according to the specific design of the catheter, and are omitted from the present discussion.

In a typical procedure to open the body lumen 18, the guide wire 23 is advanced through the patient's vascular system by well known methods so that the distal end of the guide wire is advanced past the location for the placement of the balloon or stent in the body lumen 18. The cardiologist may then perform an angioplasty procedure or other procedure (i.e., atherectomy) in order to open the vessel and remodel the diseased area. This involves inflating the balloon 14 so that the walls of the body lumen 18 are expanded, and any plaque or obstruction is cleared from the center of the artery. In some cases, this procedure is followed by the implantation of a vascular stent. A stent delivery catheter assembly similar to 10 is advanced over the guide wire 23 so that the stent is positioned in the target area. The balloon 14 is inflated so that it expands radially outwardly and in turn expands the stent radially outwardly until the stent bears against the vessel wall of the body lumen 18. The balloon 14 is then deflated and the catheter withdrawn from the patient's vascular system, leaving the stent in place to dilate the body lumen. The guide wire 23 typically is left in the lumen for post-dilatation procedures, if any, and subsequently is withdrawn from the patient's vascular system. As depicted in FIG. 2, the balloon 14 is in its delivery configuration positioned adjacent a portion of the body lumen 18 to be treated, and in FIG. 4, the balloon is fully inflated to widen the diameter of the body lumen and press against the walls of the artery. When the balloon 14 is then deflated and removed, the walls of the artery will remain open.

FIG. 2 illustrates an enlarged view, partially cut away, of the balloon 14 showing a series of markers 26 arranged circumferentially around the inner tubular member 20 at axially spaced intervals. The markers 26 can be formed from painted lines using a radiopaque paint that can be viewed under fluoroscopy. If the markers 26 can be placed at equal intervals, and even more precisely, at intervals corresponding to units of measure such as, for example, millimeters, then the physician can use the balloon once placed adjacent the lesion or blockage to accurately “measure” the obstruction 30 with the balloon 14 as if the physician had a ruler in the body lumen. This measurement can be used to determine what size balloon or stent is appropriate should such a device be needed, as well as provide information on the relative position of the lesion with respect to other body physiology. That is, measurements can be made with respect to arterial branches, lumen openings, spacings of perceived weaknesses, etc. that can be used to determine the appropriate selection of stent, procedure, and the like.

The shape or length of the markers 26 can vary, as long as they can serve as a measure device for evaluating objects adjacent the balloon 14 under fluoroscopy or other visual means. The markers 26, if they are spaced apart by a known and recognized measurement such as a millimeter, eighth inch, etc., can be observed under the fluoroscope or other means and can be used to precisely locate the catheter, the balloon 14, and the stent if present and measure the length of any obstructions 30, lesions, tears or damage, and the like. This information can be critical to a physician in selecting the appropriate balloon, stent, or other equipment.

The radiopaque material that may be used to create the divisional markers can be selected from platinum-iridium alloys, barium sulfate, or other known materials used in the art to establish indicators that can be seen under fluoroscopy. The platinum-iridium material can be formed into bands that are implanted into or crimped onto the tubular member, or painted directly on to its outer surface. In yet an alternative embodiment, the markers are incorporated into the guidewire 23 of the catheter.

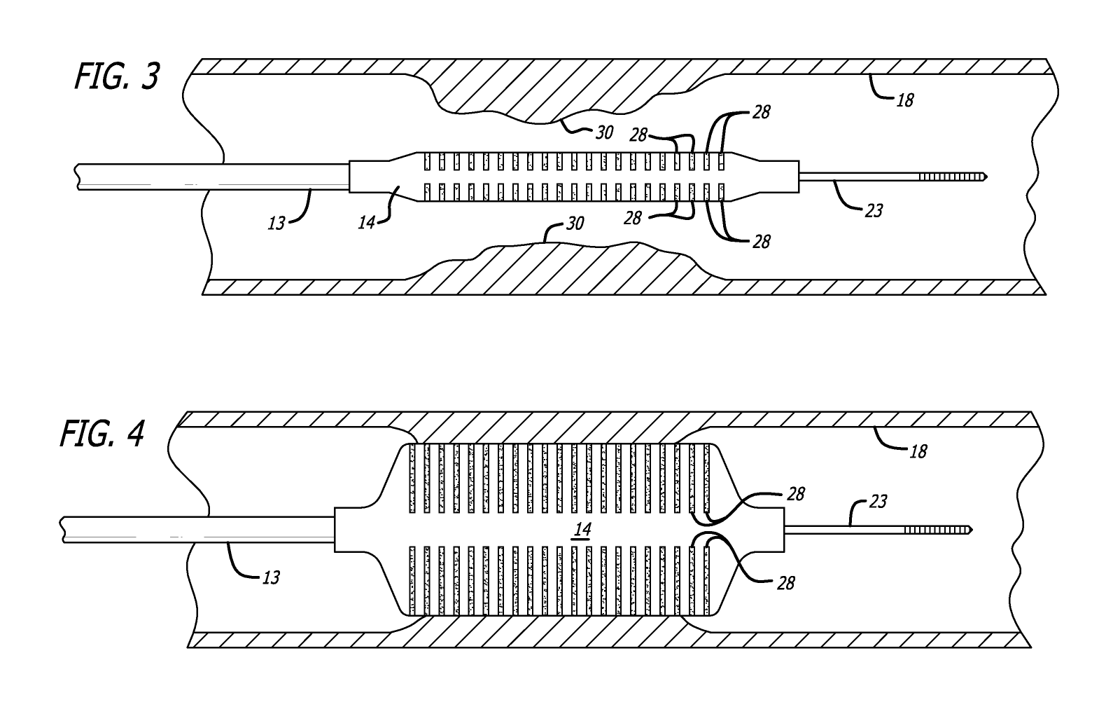

Another embodiment of the invention is depicted in FIGS. 3 and 4, where the markers 28 are placed directly on the outer surface of the balloon 14. If the markers 28 are established to be, for example, one millimeter apart, the balloon 14 can be advanced into the body lumen 18 adjacent the obstruction 30. A priori, the physician may not have a good understanding of the length of the obstruction and what size stent may be required to completely address the obstruction 30. The balloon 14 is maneuvered into place adjacent the obstruction 30, such that the markers 28 (or markers 26 for the embodiment of FIGS. 1 and 2) are aligned with the obstruction. The physician notes that the markers 28 are one millimeter apart, and that the lesion spans twenty-one markers, or twenty millimeters in length. Thus, if a stent is to be implanted to completely address the obstruction 30, it must be at least 20 millimeters. This type of accuracy is pivotal in such procedures and was heretofore unavailable without the present invention. The balloon 14 can be withdrawn after inflation and deflation and a stent can be implanted at the site with the dimensions obtained from the measurement with the markers 26, ensuring that the full length of the obstruction is adequately accounted for.

The benefit of using the tubular member 20 (or the guidewire 23) rather than the balloon 14 to locate the markers is that there is no distortion of the tubular member 19 when the balloon is inflated, whereas the balloon can undergo some elongation as it inflates, or distortion as it is crimped, which would disturb the actual distances used to measure the lesions. Thus, in the case of markers 28 directly on the balloon surface, the measurement must either be made prior to inflation of the balloon, or the balloon must be made of a low compliant material that inflates without axial elongation to preserve the proportion of the markers and maintain an accurate “ruler.”

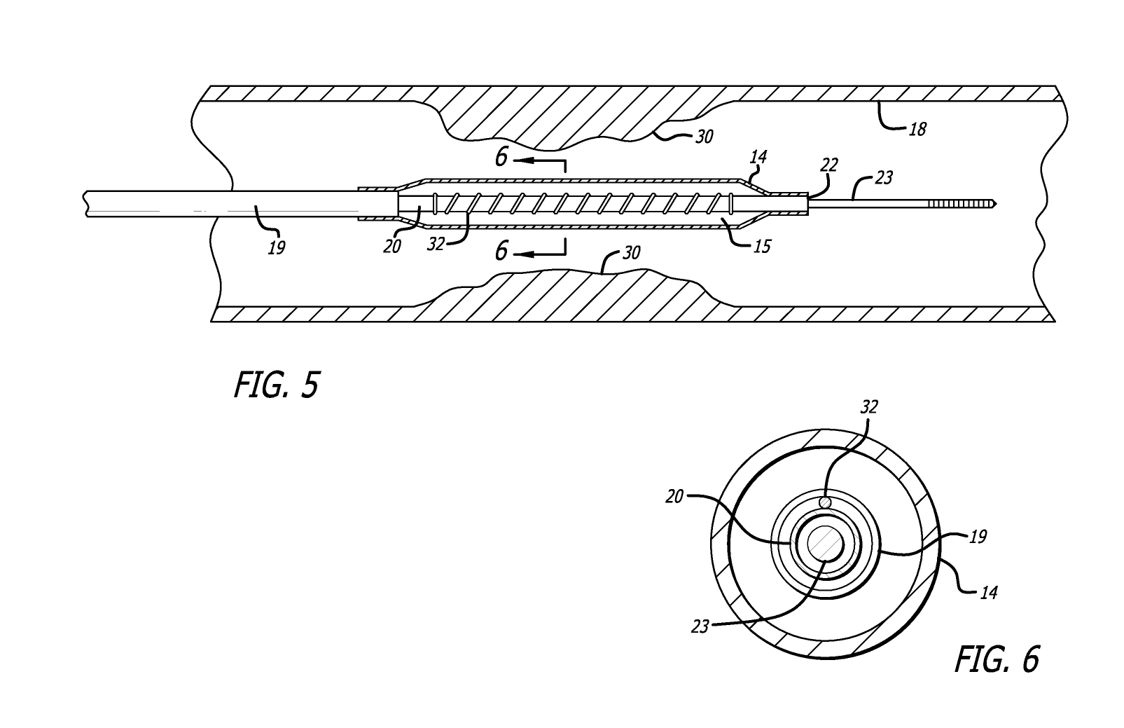

FIGS. 5 and 6 illustrate another embodiment of the present invention, where a radiopaque ribbon or thread is wrapped around the inner member so as to provide a predetermined spacing between each successive winding. That is, the pitch of the spiral wound radiopaque ribbon is constant and selected so as to provide a continuous ruler three hundred and sixty degrees around the inner member. This allows the physician to view the spacings from any angle and provides a more effective means of evaluating a lesion's length.

Similarly, markings can comprise raised radiopaque beads that could provide greater contrast due to the three dimensional nature of the beads. This can be helpful is evaluating certain structures outside the catheter body where it is a challenge to obtain a normal view. The beads can be seen from a wider set of angles, making them preferable in some circumstances. The beads, like the ribbon of FIG. 5, can be located anywhere on the catheter body including the balloon itself. Having the measurement markings on the balloon has the benefit of proximity to the lesion itself, thereby reducing the error due to parallax, but care is needed to ensure that there is no distortion to the markings as the balloon is inflated if measurements are to be taken post-inflation.

While particular forms of the invention have been illustrated and described, it will be apparent to those skilled in the art that various modifications can be made without departing from the spirit and scope of the invention. Accordingly, it is not intended that the invention be limited except by the appended claims.

What is claimed:

1. A catheter, comprising:
an elongate tubular member having a first lumen for a guidewire, and a second lumen for inflating a balloon member;
a balloon member disposed on a distal portion of the elongate tubular member, the balloon member having a working body section and a proximal and a distal taper section; and
a plurality of visual radiopaque markers spaced axially along the catheter, where a spacing of the markers correspond with a discrete unit of measurement.

2. The catheter of claim 1, wherein the discrete unit of measurement is a millimeter.

3. The catheter of claim 1, wherein the markers are painted on the balloon using a radiopaque paint.

4. The catheter of claim 1, wherein the markers are embedded into the balloon.

5. The catheter of claim 1, wherein the markers are formed from a platinum-iridium alloy.

6. The catheter of claim 1, wherein the markers are formed from barium sulfate.

7. The catheter of claim 1, wherein the markers are formed into a band that is crimped onto the balloon.

8. The catheter of claim 1, wherein the markers are located on an inner shaft forming part of the inflation lumen.

9. The catheter of claim 1, wherein the markers are located on an outer surface of the inflation lumen.

10. The catheter of claim 1, wherein the markers are located on a guidewire passing through the balloon.

11. The catheter of claim 1, wherein the visual markers can be observed under fluoroscopy.

12. A method for measuring an object in a body lumen comprising:
providing a catheter having a plurality of visual markers spaced at discrete and uniform intervals, the intervals corresponding to a unit of measure;
maneuvering the catheter into the body lumen adjacent the object to be measured; and
visually comparing a length of the object to the number of markers on the catheter to determine how many units of measure the object extends for.

13. The method of claim 12, wherein the visually comparing is conducted using a fluoroscope.

14. The method of claim 12, wherein the unit of measure is a millimeter.

15. The method of claim 12, wherein the visual markers are formed from a platinum-iridium alloy.

16. The method of claim 12, wherein the visual markers are formed from barium sulfate.

17. The method of claim 12, wherein the markers are disposed on an inner tubular member that forms part of an inflation lumen.

18. The method of claim 12, wherein the markers are disposed on an outer surface of a balloon.

19. The method of claim 12, wherein the markers are disposed on a guidewire.

20. The method of claim 12, wherein the markers are painted onto a surface of the catheter.

21. The method of claim 12, wherein the markers are embedded into the body of the catheter.

22. A catheter, comprising:
an elongate tubular member having a first lumen for a guidewire, and a second lumen for inflating a balloon member;
a balloon member disposed on a distal portion of the elongate tubular member, the balloon member having a working body section and a proximal and a distal taper section; and
a radiopaque ribbon wound around the elongate tubular member so as to have a spacing between successive windings corresponding to a discrete unit of measurement.
